(19) United States
(12) Patent Application Publication
(10) Pub. No.: US 2024/0423659 A1
(43) Pub. Date: Dec. 26, 2024

(54) A SKIN MINCING DEVICE

(71) Applicant: Pacify Medical Technologies Private Limited, Thane (IN)

(72) Inventors: Saiprasad Poyarekar, Mumbai (IN); Anmol Zimare, Mumbai (IN); Rayyan Ramrajkar, Mumbai (IN)

(21) Appl. No.: 18/699,276

(22) PCT Filed: Oct. 7, 2022
(86) PCT No.: PCT/IN2022/050895

§ 371 Date: Apr. 6, 2024

(30) Foreign Application Priority Data
Oct. 7, 2021 (IN) 202121045572

(51) Int. Cl. A61B 17/30; A61B 17/00; A61B 17/322

(52) U.S. Cl. CPC A61B 17/30; A61B 2017/00747; A61B 2017/3225

(57) ABSTRACT

There is provided a device for processing harvested skin. The device includes a bed 104 to hold a skin graft held over a template 110 and a support part 102 with multiple cutting slots 114 running through a length of the support part 102. The support part 102 is positioned over the bed 104 to apply a uniform force to press the support part 102 against the skin graft. A first cut of the skin graft is performed with a scalpel blade by locking the support part 102 at a zero position against the bed 104 and a second cut of the skin graft is performed by lifting the support part 102 facilitated by a lifting element and locking the support part 102 by rotating the support part 102 to 90 degrees from the zero position with respect to the bed 102 to obtain a square shaped skin graft pieces.

### TECHNICAL FIELD

The embodiments herein generally relate to a device for surgical procedures, more particularly to a skin mincing device for processing harvested or artificial skin to be used to cover wounds.

### BACKGROUND

Skin grafting is required if there is loss of skin due to accidents, certain diseases, or wounds due to burns. It has been a practice to take a skin graft from a healthy site on the body and transfer it to the wound site. But this means creating another wound and the dimension of the healthy skin to be taken is in interest to be taken in limited quantity. Usually, the skin taken is worked on to improve the surface area that can be covered using that amount of skin. There has been a known practice to stretch the skin using its elastic properties and cutting them into small pieces and then apply it to wounds. But the problem with existing cutting methods for the skin is that it's not possible to cut the skin into uniform small pieces. As the dimension of the skin pieces gets smaller, there is increased loss of shape of the skin piece as the elastic properties of the skin make it easily recoilable. And, the result ends up being a paste-like consistency of skin instead of uniform pieces. It is hard to apply on wounds uniformly in a single layer.

Accordingly, there is a need for an improved skin mincing device and method to avoid aforementioned complications.

### SUMMARY OF THE INVENTION

According to the first aspect, a device for mincing a skin graft is provided. The device includes a bed comprising a placement area. The placement area is configured to receive a template holding the skin graft. The template comprises a number of pillar-like structures that comprises cavities between the pillars. A support part is configured to place over the bed holding the skin graft on the placement area. Multiple cutting guides and multiple cutting slots run parallel through the length of the support part. The multiple cutting guides and multiple cutting slots are positioned equidistant from each other. Multiple cutting guides on support part are positioned to guide the user and identify where the cutting has been done. A scalpel blade handle comprising one or more blades are guided to perform (i) a first cut of the skin graft by locking the support part at a zero position against the bed: and (ii) a second cut of the skin graft by lifting the support part facilitated by a lifting element that facilitates vertical motion of the support part when rotated either clockwise or anticlockwise and locking the support part by rotating the support part to 90 degrees from the zero position with respect to the bed to obtain a square shaped skin graft pieces. The scalpel blade handle is guided to cut the skin graft through the multiple cutting slots using the multiple cutting guides of the support part.

Optionally, the multiple cutting slots in the support part are positioned corresponding to the one or more scalpel blades to facilitate a single stroke cut.

Optionally, the bed comprises one or more bed slots provided at one or more corners or edges of the bed and the support part comprises one or more support part slots provided at one or more corners or edges of the support part.

Optionally, one or more fasteners are configured to lock the support part with the bed by passing through the one or more support part slots and the one or more bed slots. The one or more fasteners are configured to lock the support part against the bed holding the skin graft on the placement area.

Optionally, the lifting element is provided on a first ring that is locked securely with the bed. The lifting element comprises four springs mounted on the first ring at 90 degrees apart (i) to keep the skin graft intact on the placement area, and (ii) to push the support part gradually upward during unlocking of the support part to perform the second cut.

Optionally, the template has a non-stick surface to allow easy removal of the skin pieces. The non-stick surface is provided in the form of non-stick material comprising teflon, non-stick coatings, non-stick papers, non-stick chemicals, or powders.

Optionally, the device is configured to perform mincing of the skin graft comprising variable thickness.

According to the second aspect, a method of mincing a skin graft is provided. The method includes placing a skin graft on a placement area of a bed. The placement area is configured to receive a template holding the skin graft. The template comprises a number of pillar-like structures that comprise cavities between the pillars. The method includes placing a support part over the bed holding the skin graft on the placement area. Multiple cutting guides and multiple cutting slots run parallel through a length of the support part. The multiple cutting guides and the multiple cutting slots are positioned equidistant from each other. The method includes guiding a scalpel blade handle comprising one or more blades to perform (i) a first cut of the skin graft by locking the support part at a zero position against the bed; and (ii) a second cut of the skin graft by lifting the support part facilitated by a lifting element that facilitates vertical motion of the support part when rotated either clockwise or anticlockwise and locking the support part by rotating the support part to 90 degrees from the zero position with respect to the bed to obtain a square shaped skin graft pieces. The scalpel blade handle is guided to cut the skin graft through the multiple cutting slots using the multiple cutting guides of the support part.

Optionally, the bed comprises one or more bed slots provided at one or more corners or edges of the bed and the support part comprises one or more support part slots provided at one or more corners or edges of the support part.

Optionally, one or more fasteners are configured to lock the support part with the bed by passing through the one or more support part slots and the one or more bed slots. The one or more fasteners are configured to lock the support part against the bed holding the skin graft on the placement area.

Optionally, the lifting element is provided on a first ring that is locked securely with the bed. The lifting element comprises four springs mounted on the first ring at 90 degrees apart (i) to keep the skin graft intact on the placement area, and (ii) to push the support part gradually upward during unlocking of the support part to perform the second cut.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates an assembly view of a skin mincing device, according to an embodiment herein;

FIG. 1A illustrates an exploded view of a skin mincing device, according to an embodiment herein;

FIG. 2 illustrates a scalpel blade handle corresponding to the skin mincing device of FIG. 1, according to an embodiment herein;

FIG. 3 illustrates uniformly cut skin graft pieces at the skin mincing device of FIG. 1, according to an embodiment herein;

FIG. 4A illustrates a circular-shaped skin mincing device, according to an embodiment herein;

FIG. 4B illustrates an exploded view of the circular shaped skin mincing device of FIG. 5B, according to an embodiment herein;

FIG. 5 illustrates a flow diagram of a method of using the skin mincing device of FIG. 1 to obtain uniformly cut skin graft pieces according to an embodiment herein; and

FIGS. 6A and 6B illustrate a flow diagram of a method of using the circular-shaped skin mincing device of FIG. 4A to obtain uniformly cut skin graft pieces according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

As mentioned, there remains a need for a skin mincing device that ensures clean and efficient cutting of the skin into small enough pieces to cover a wound area without damaging the skin by undue stress or strain.

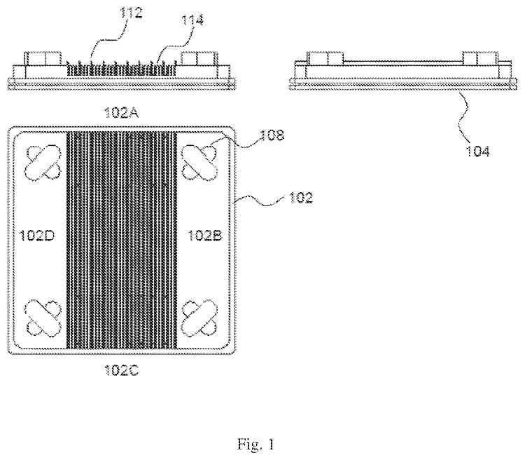

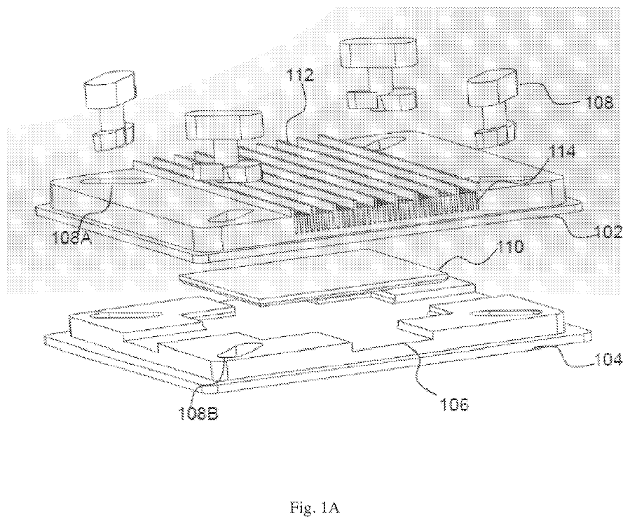

Referring now to the drawings, and more particularly to FIGS. 1 through 4, where similar reference characters denote corresponding features consistently throughout the figures, preferred embodiments are shown.

FIG. 1 illustrates an assembly view of a skin mincing device, according to an embodiment herein. The skin mincing device includes a support part 102, one or more fasteners 108 at four corners of the support part 102, parallel cutting guides 112 and cutting slots 114 running through a length of the support part 102 between two cutting sides 102A and 102C, 102B and 102D are non-cutting sides of the support part 102. The skin mincing device also includes a side view that shows a bed 104 on which the support part 102 is fitted. The fasteners 108 may be rotated about 30 to 180 degrees in either direction to lock and unlock the assembly. The support part 102 is completely detachable from the bed 104. FIG. 1A illustrates an exploded view of a skin mincing device, according to an embodiment herein. The view includes the support part 102, the bed 104 with a placement area 106, the one or more fasteners 108, one or more fastener slots in the support part 108A, one or more fastener slots in the bed 108B, a skin graft to be placed on a template 110 for cutting, the parallel cutting guides 112, and the cutting slots 114. The template 110 may be flat or contain a number of small pillar-like structures with dimensions equal to or less than the desired size of skin pieces. The cavity between the pillars may facilitate passage for blades to pass below the skin graft surface. The template that holds the skin graft may be flat or slotted with mesh-like structure. Water soluble bio-compatible paper or non-stick sheets such as teflon may be used as base surface on the template 110 to keep the skin which allows cutting through the material. Skin holding part, template surface has a non-stick effect in the form of non-stick material such as teflon, non-stick coatings, non-stick papers, non-stick chemicals/powders to allow easy removal of skin pieces. The template 110 may be made using a combination of metal and plastic material. The support part 104 is kept on top of the skin graft placed on the template 110 and the bed 104 to hold the skin graft in place by covering the skin graft and positioning it for cutting by using one or more fasteners 108 to apply downward force to press the support part 102 against the skin graft thereby holding the cut pieces in place, helping with the uniform cutting without recoiling of the cut skin tissue. The bed 104 and skin pressure applying portion of the support part 102 comprises non-stick material such as teflon, non-stick coatings, non-stick papers, non-stick chemicals/powders to avoid sticking of skin. The support part 104 may be made using a combination of metal and plastic material. The components of the skin mincing device may be connected using any connecting mechanism or remain free to slide in-out as free parts.

The support part 102 includes multiple cutting slots 114 running parallelly through a length of the support part 102. The cutting slots 114 may be equidistant from each other with parallel cutting guides 112 and correspond to a scalpel blade handle comprising one or more blades to pass through the cutting slots 114 to cut through the skin graft. For the first time, the scalpel blade handle is used to hold one or more blades which are passed through the skin graft through the support part 102 through the cutting slots 114, resulting in strip pieces of the skin graft on the template 110 placed on the placement area 106. In some embodiments, dermal side of the skin graft faces the bed 104 and dermis side (outer skin) faces the support part 102, ensuring that after cutting, the skin graft pieces stay on the placement area 106 of the bed 104. The skin or tissue is held in place using glue or mechanical fasteners on the template 110. The mechanical fasteners may comprise a threaded arrangement or have a curved surface to hold the skin or tissue in place.

The one or more fasteners 108 may be rotated about 30 to 180 degrees towards a first side and removed from the corresponding one or more fastener slots in the support part 108A, and from the one or more fastener slots in the bed 108B, thereby relieving the pressure on the skin graft, and allowing the support part 102 to be removed and reinstated on switching of the two non-cutting sides with the two cutting sides, by rotating the support part 102 by 90 degrees towards a second side and fixing the support part 102 using the one or more fasteners 108 in place on top of the bed 104, by rotating the one or more fasteners 108 to 90 degrees towards the first side through the one or more fastener slots in the support part 108A, and from the one or more fastener slots in the bed 108B. For the second time, the scalpel blade handle is used to hold one or more scalpel blades which are passed through the skin graft through the support part 102 through the cutting slots 114, resulting in finely cut uniform square pieces of the skin graft in the placement area 106, ready to be used. The one or more fasteners 108 may be again rotated 90 degrees towards the first side to detach from the bed 104 and the support part 104, and the support part 102 may be lifted off the bed 104. The one or more fasteners 108 may include four fasteners that are rotated about 90 degrees to apply a force of about 30 to 60N on the skin graft placed on the placement area to avoid recoiling of the skin graft.

In some embodiments, after cutting, the support part 102 is opened sideways on one of the two non-cutting sides, holding, and lifting one of the two non-cutting sides, and letting the opposite noncutting side rest on the bed 104, like a book, enabling the cut skin graft pieces to stay in the placement area 106 of the bed 104. The support part 102 has two cutting sides and two non-cutting sides facing each other as shown.

Optionally, there are at least four cutting slots 114 in the support part 102 which correspond to four scalpel blades in the scalpel blade handle. Optionally, the distance between the slots 108 is 0.3 to 2.5 millimetres. Optionally the distance between the slots 108 is 2 millimetres. The support part 102 is rotated to 90 degrees and fixed again with the one or more fasteners 108 on the bed 104 and the skin graft. The scalpel blade handle is used to hold one or more scalpel blades which are passed on skin graft from 90 degrees to the first cutting through the support part 102 through the cutting slots 114. The support part 102 has parallel cutting guides 112 to ensure equidistant strokes. The blade holder is fixed in angle with respect to support's top surface, also fixing depth of cut. The device may also contain, a structure to restrain the blade holder from moving upward. Blades can also be mounted in reverse structure so that the resultant action is pulling the skin graft upwards. The shape of the blade may be a circular, a straight oval or a roller type. Multiple blades with more than one blade may be assembled to work as one cutting tool. Blades are provided with guides to ensure fix path of motion. Blades may have a heating arrangement to heat multi-blade assembly, to smoothly and accurately cut the skin into pieces with the least amount of effort. The heating arrangement includes a heating element that remains inside a holding feature of the blade holder. The skin mincing device employs ultrasonic cutting which uses precise vibrations to cut the skin.

The skin tissues are cut into equal square pieces. The cut skin tissues are transferred to a sterile container for storage or taken for application on the wound site.

For general skin grafting purposes, it is desirable to extend the skin tissue as much as possible. By managing the pressure and integrity of the skin graft tissues after being cut, the skin mincing device enables increased usability and efficiency in using the skin graft to cover a wound area of the patient.

For split thickness skin grafts that are generally used, lower pressures such as 50 mm Hg may be tolerated without compromise of skin graft incorporation. As per the experimental data of Management of skin grafts using negative pressure therapy: the effect of varied pressure on skin graft incorporation.

In some embodiments, the size of the skin graft may be in a range of 50 mm×50 mm×0.8 mm to 5 mm×5 mm×2 mm (Width×Length×Thickness).

Optionally, the size of the skin graft may include a large group of size.

The skin being an elastic material, is subject to the mechanical laws defining its properties. The modulus of longitudinal elasticity—the Young's modulus (E)—defines the relation between stress (σ) and strain (ε) in the skin.

The modulus characterizes skin resistance to elastic elongation. This dependence is generally presented by the Hooke's Law: ε=E/σ. In the elastic range the relationship between stress and strain is linear and the factor of proportionality is expressed by the Young's modulus. The unit of Young's modulus is the Pascal (Pa, N/m2).

The Young's modulus is defined as: E=tg α. In the literature, the Young's modulus (E) of the skin fluctuates between 0.42 MPa and 0.85 MPa for the torsion tests, between 4.6 MPa and 20 MPa when extracting stress values obtained in tests carried by means of mechanical equipment, and between 0.05 MPa and 0.15 MPa in the suction tests. The skin's natural elasticity means when the skin is stretched and cut, it will tend to recoil, making it difficult to get uniformly cut small pieces without external pressure. The pressure may be adjusted between 30-60N to avoid overstretching or under supporting the cutting of the skin tissue of the skin graft. Some discrepancies in the results may correspond to the changes occurring in the skin during the process of aging, as well as the differences in the skin properties depending on its anatomic location.

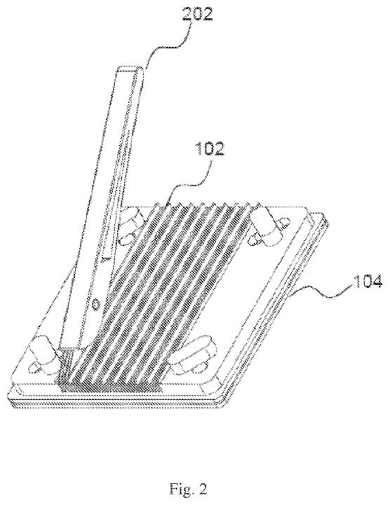

To avoid recoiling of the skin when cut into shorter pieces, the skin graft responds well to the pressure of 50N applied with the help of the fasteners 108 fitting the support part 102 onto the bed 104 while the skin graft is placed in-between at the placement area 106, where the skin graft is in contact with the bed 104 and the support part 102 from either side, holding the skin graft in place even after cutting into small pieces. FIG. 2 illustrates a scalpel blade handle corresponding to the skin mincing device of FIG. 1, according to an embodiment herein. The skin graft is placed in the placement area 106 (not shown), the fasteners 108 lock the support part 102 to the bed 104. A scalpel blade handle 202 is guided to cut through the cutting slots 114 using the parallel cutting guides 112 which hold the skin graft in place. The scalpel blade handle 202 comprises one or more blades of the skin mincing device that are enabled to perform (i) a first cut of the skin graft by locking the support part 102 at a zero position against the bed 104; and (ii) a second cut of the skin graft by lifting the support part 102 facilitated by a lifting element that facilitates the vertical motion of the support part 102 when rotated either clockwise or anticlockwise and locking the support part by rotating the support part to 90 degree from the zero position with respect to the bed 104 to obtain a square shaped skin graft pieces. The scalpel blade handle 202 is enabled to cut the skin graft through the multiple cutting slots using the multiple cutting guides of the support part 102. The multiple cutting slots 114 in the support part 102 are positioned corresponding to the one or more scalpel blades to facilitate a single stroke cut. The bed 104 comprises one or more bed slots provided at one or more corners or edges of the bed and the support part comprises one or more support part slots provided at one or more corners or edges of the support part.

There may be at least two blades connected to the scalpel blade handle 202, corresponding to four cutting slots 114 between the parallel cutting guides 112. The cutting strokes may be made several times corresponding to each of track between the parallel cutting guides 112, cutting until the entire placement area 106 is covered. The fasteners 108 may be unlocked and the support part 102 is rotated 90 degrees and fixed again on the bed 104 by locking with the fasteners 108. The scalpel blade handle 202 is used again for cutting in a similar way but now at a 90-degree angle to the first cutting. Once the entire placement area is covered, the fasteners are used to unlock the support part 102 from the bed 104. On opening the support part 102, finely and uniformly cut skin graft pieces are found on the placement area 106. In an embodiment, a scalpel blade that is used is of size 21. A liquid is poured on the template and the finely cut uniform square pieces of the skin graft are collected in a dish. The square pieces of the skin graft are of 1 mm×1 mm dimensions.

The device comprises one or more fasteners 108 that are configured to lock the support part 102 with the bed 104 by passing through the one or more support part slots and the one or more bed slots. The one or more fasteners 108 are configured to lock the support part against the bed holding the skin graft on the placement area 106. The lifting element is provided on a first ring that is locked securely with the bed 104. The lifting element comprises four springs mounted on the first ring at 90 degrees apart to (i) keep the skin graft intact on the placement area 106, and (ii) push the support part 102 gradually upward during unlocking of the support part 102 to perform the second cut. The template 110 has a non-stick surface to allow easy removal of the skin pieces. The non-stick surface is provided in the form of non-stick material comprising teflon, non-stick coatings, non-stick papers, non-stick chemicals, or powders. The device is configured to perform mincing of the skin graft with variable thickness

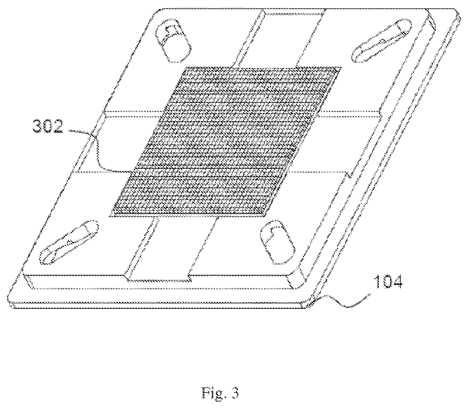

FIG. 3 illustrates uniformly cut skin graft pieces at the skin mincing device of FIG. 1, according to an embodiment herein. As the cutting happens in two directions, a first cut of the skin graft is performed by locking the support part 102 at a zero position and then a second cut is performed by changing the direction of the support part 102 by rotating 90 degrees from the zero position, resulting in finely and uniformly minced square skin graft pieces.

One or more blades of the skin mincing device are enabled to perform (i) a first cut of the skin graft by locking the support part at a zero position against the bed; and (ii) a second cut of the skin graft by lifting the support part facilitated by a lifting element that facilitates the vertical motion of the support part when rotated either clockwise or anticlockwise and locking the support part by rotating the support part to 90 degree from the zero position with respect to the bed to obtain a square shaped skin graft pieces.

For example, during Burn surgeries, the surgeon takes a skin graft from a healthy region of patient's body with the help of a surgical knife or dermatome and covers the burn area of the patient with the skin graft. If the burn wounds are extensive, it is very helpful to cut the skin graft into very small pieces and spread them evenly over the wound area. Generally, Regeneration of wound starts from healthy region. Hence, wound healing starts from the surrounding to the centre. Healthy skin tissues act as seeds for regeneration. By using this principle, it is possible to spray-cut skin graft tissues on the wound area. The cut skin graft tissue settles down on the surface of wound to cover the wound and regeneration of the wound starts from the surrounding of the skin graft tissues. The wound healing starts from different points inside the wound to the surrounding along with the recovery which is already going on from the surrounding to the centre. Consequently, this accelerates the recovery of the wound. An air-assisted nozzle may be used to spray the cut skin graft tissues evenly. The pressure may be maintained at two bars above the atmospheric pressure. A special nozzle corresponding to the cut skin graft tissue size achieved using the skin mincing device may be used to spray the solid skin graft tissue on the wound, covering a surface area.

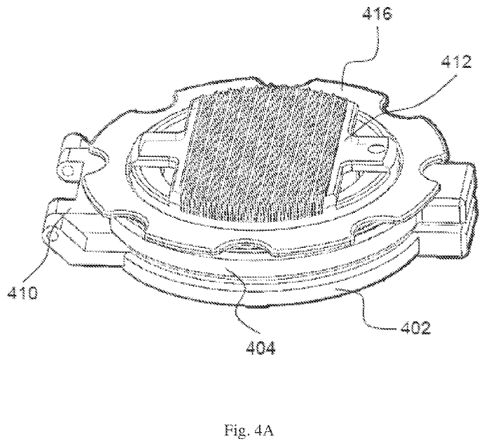

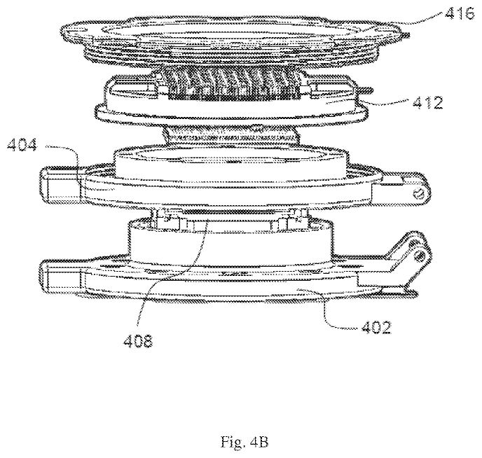

In case of wounds, the healthy area available on the patient body provides the skin graft which is then very effectively used by the help of the skin mincing device to be cut into small pieces and in a sterile environment, may be sprayed on the wound, covering an area, to bring effective recovery and to improve the prognosis of the burn patient. FIG. 4A illustrates a circular-shaped skin mincing device, according to an embodiment herein. The circular-shaped skin mincing device includes a circular bed 402, a first ring 404, a template, a placement area, a clamp 410, a support part 412 provided with an index plunger, a second ring 416. The first ring 404 is attached to the circular bed 402 using a hinge on a first side. The clamp 410 is attached to a second side of the first ring 404. The first ring 404 is locked securely with the circular bed 402 with the clamp 410 to form a base portion. Four Springs are mounted on the ring part at 90 degrees apart. These springs are used to push gradually the support part 412 upward. The user is not required to lift the support part 412 manually. Due to the action of the springs the support part 412 will automatically move-up facilitating rotation of the support part 412 by pulling the index plunger.

A skin graft is obtained by cutting skin corresponding to the size of the template with a scalpel. A water-soluble paper is placed on the skin graft to provide non-stick effect. The Clamp 410 is attached to hold the first ring 404 and circular bed 402 in contact for holding the water-soluble paper. The water-soluble paper is placed over the skin in a position to act as a non-stick agent that allows cutting. The skin graft is placed on the template. The template comprising the skin graft is placed on the placement area. The support part 412 is placed over the base in zero position using the index plunger 414 the degrees of rotation. The index plunger is used to locate the second ring 416 that is placed over the support part 412 and rotated to secure the skin graft in a position to facilitate cutting in the placement area. Threads are used for tightening by rotation of second ring 416.

The support part 412 includes multiple cutting slots running parallelly through a length of the support part 102. The cutting slots 114 may be equidistant from each other with parallel cutting guides. A scalpel blade is used to perform a first cut on the skin graft through the cutting slots. The scalpel blade passes through the cutting slots to cut through the skin graft. The dermal side of the skin graft may be kept facing the circular bed 402 and the dermis side (outer skin) may be kept facing the support part 412, ensuring that after cutting, the skin graft pieces stay on the placement area of the circular bed 402. A first cut is performed on the skin graft with the scalpel blade with the support part 412 being is placed over the base at the zero position. The second ring 416 placed over the support part 412 is loosened to rotate the support part 412 to 90 degrees. The second ring 416 is secured over the support part 412 after rotating the support part 412 to 90 degrees. The 90 degrees rotation is performed by the index plunger settled in a hole drilled on the first ring 404. A second cut is performed on the skin graft with the scalpel after rotating the support part 412 to 90 degrees from the zero position to obtain finely cut uniform square pieces of the skin graft on the template placed on the placement area. A liquid is poured on the template and the finely cut uniform square pieces of the skin graft are collected in a dish. The square pieces of the skin graft are of 1 mm×1 mm dimensions.

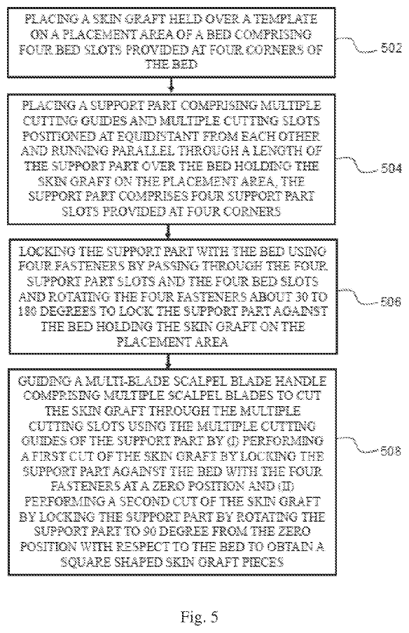

FIG. 4B illustrates an exploded view of the circular-shaped skin mincing device of FIG. 5B, according to an embodiment herein. The exploded view of the circular-shaped skin mincing device includes the circular bed 402, the first ring 404, a placement area 408, a support part 412, and a second ring 416. The functioning of the components has been described above.

FIG. 5 illustrates a flow diagram of a method of using the skin mincing device of FIG. 1 to obtain uniformly cut skin graft pieces according to an embodiment herein. At a step 502, the method includes placing a skin graft held over a template on a placement area of a bed comprising four bed slots provided at four corners or edges of the bed. At a step 504, the method includes placing a support part comprising multiple cutting guides and multiple cutting slots positioned at equidistant from each other and running parallel through a length of the support part over the bed holding the skin graft on the placement area, the support part comprises four support part slots provided at four corners or edges. At a step 506, the method includes locking the support part with the bed using four fasteners by passing through the four support part slots and the four bed slots and rotating the four fasteners about 30 to 180 degrees to lock the support part against the bed holding the skin graft on the placement area. At a step 508, the method includes guiding a scalpel blade handle comprising one or more scalpel blades to cut the skin graft through the multiple cutting slots using the multiple cutting guides of the support part by (i) performing a first cut of the skin graft by locking the support part against the bed with the four fasteners at a zero position and (ii) performing a second cut of the skin graft by locking the support part by rotating the support part to 90 degrees from the zero position with respect to the bed to obtain a square shaped skin graft pieces.

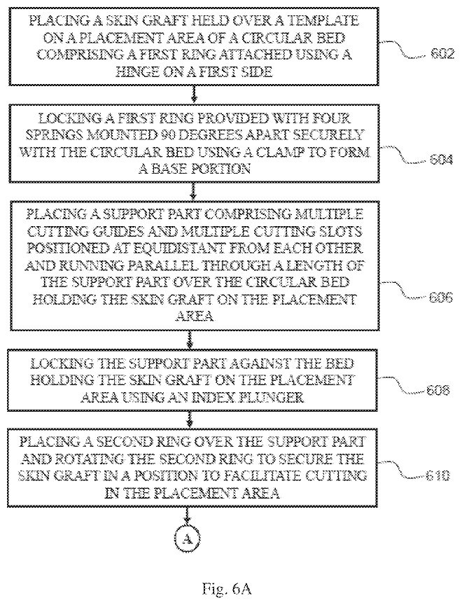

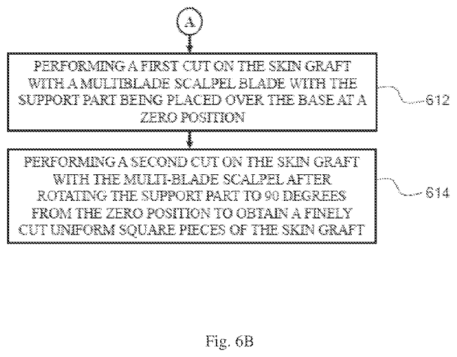

FIGS. 6A and 6B illustrate a flow diagram of a method of using the circular-shaped skin mincing device of FIG. 4A to obtain uniformly cut skin graft pieces according to an embodiment herein. At a step 602, the method includes placing a skin graft held over a template on a placement area of a circular bed comprising a first ring attached using a hinge on a first side. At a step 604, the method includes locking a first ring provided with four springs mounted 90 degrees apart securely with the circular bed using a clamp to form a base portion. At a step 606, the method includes placing a support part comprising multiple cutting guides and multiple cutting slots positioned at equidistant from each other and running parallel through a length of the support part over the circular bed holding the skin graft on the placement area. At a step 608, the method includes locking the support part against the bed holding the skin graft on the placement area using an index plunger. At a step 610, the method includes placing a second ring over the support part and rotating the second ring to secure the skin graft in a position to facilitate cutting in the placement area. At a step 612, the method includes performing a first cut on the skin graft with a scalpel blade with the support part being placed over the base at a zero position. At a step 614, the method includes performing a second cut on the skin graft with the scalpel blade after rotating the support part to 90 degrees from the zero position to obtain finely cut uniform square pieces of the skin graft.

The advantage of using the skin mincing device includes achieving very finely and uniformly cut skin graft pieces which is easy to handle and allows the surgeon to spread the skin tissue evenly over an area of a wound. This may prevent secondary infection and cover the over-burned area, even if the healthy skin graft size is very limited. It is possible to improve the prognosis, and to handle the healthy skin tissue from the patient very efficiently in a sterile environment, with a cost-effective skin mincing device. A solid particle spray gun may be further used to spray the uniformly and finely cut skin graft pieces over a wound area.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

We claim:

1. A device for mincing a skin graft, wherein the device comprises:
a bed (104) comprising a placement area (106), wherein the placement area (106) is configured to receive a template (110) holding the skin graft, wherein the template (110) comprises a number of pillar-like structures that comprise cavities between the pillars:
a support part (102) that is configured to place over the bed (104) holding the skin graft on the placement area (106), wherein multiple cutting guides (112) and multiple cutting slots (114) run parallel through a length of the support part (102), wherein the multiple cutting guides and multiple cutting slots are positioned equidistant from each other;
a scalpel blade handle (202) comprising one or more blades, characterized in that, wherein the one or more blades are enabled to perform (i) a first cut of the skin graft by locking the support part (102) at a zero position against the bed (104); and (ii) a second cut of the skin graft by lifting the support part (102) facilitated by a lifting element that facilitates vertical motion of the support part (102) when rotated either clockwise or anticlockwise and locking the support part (102) by rotating the support part to 90 degree from the zero position with respect to the bed (104) to obtain a square shaped skin graft pieces, wherein the scalpel blade handle (202) is enabled to cut the skin graft through the multiple cutting slots (114) using the multiple cutting guides (112) of the support part (102).

2. The device as claimed in claim 1, wherein the multiple cutting slots (114) in the support part are positioned corresponding to the one or more blades to facilitate a single stroke cut.

3. The device as claimed in claim 1, wherein the bed (104) comprises one or more bed slots provided at one or more corners or edges of the bed (104) and the support part (102) comprises one or more support part slots provided at one or more corners or edges of the support part (102).

4. The device as claimed in claim 3, wherein the device comprises one or more fasteners (108) that are configured to lock the support part (102) with the bed (104) by passing through the one or more support part slots and the one or more bed slots, wherein the one or more fasteners (108) are configured to lock the support part (102) against the bed (104) holding the skin graft on the placement area (106).

5. The device as claimed in claim 1, wherein the lifting element is provided on a first ring that is locked securely with the bed (104), wherein the lifting element comprises four springs mounted on the first ring at 90 degrees apart to (i) keep the skin graft intact on the placement area (106), and (ii) push the support part (102) gradually upward during unlocking of the support part (102) to perform the second cut.

6. The device as claimed in claim 1, wherein the template (110) has a non-stick surface to allow easy removal of the skin pieces, wherein the non-stick surface is provided in the form of non-stick material comprising teflon, non-stick coatings, non-stick papers, non-stick chemicals, or powders.

7. The device as claimed in claim 1, wherein the device is configured to perform mincing of the skin graft with variable thickness.

8. A method of mincing a skin graft, wherein the method comprises the steps of:
placing a skin graft on a placement area (106) of a bed (104), wherein the placement area (106) is configured to receive a template (110) holding the skin graft, wherein the template (110) comprises a number of pillar-like structures that comprise cavities between the pillars;
placing a support part (102) over the bed (104) holding the skin graft on the placement area (106), wherein multiple cutting guides (112) and multiple cutting slots (114) run parallel through a length of the support part (102), wherein the multiple cutting guides (112) and the multiple cutting slots (114) are positioned at equidistant from each other;
characterized in that, enabling a scalpel blade handle (202) comprising one or more blades to perform (i) a first cut of the skin graft by locking the support part (102) at a zero position against the bed (104); and (ii) a second cut of the skin graft by lifting the support part (102) facilitated by a lifting element that facilitates vertical motion of the support part (102) when rotated either clockwise or anticlockwise and locking the support part (102) by rotating the support part to 90 degrees from the zero position with respect to the bed (104) to obtain a square shaped skin graft pieces, wherein the scalpel blade handle (202) is guided to cut the skin graft through the multiple cutting slots (1a4) using the multiple cutting guides (112) of the support part.

9. The method as claimed in claim 8, wherein the bed (104) comprises one or more bed slots provided at one or more corners or edges of the bed (104) and the support part (102) comprises one or more support part slots provided at one or more corners or edges of the support part (104).

10. The method as claimed in claim 9, wherein one or more fasteners (108) are configured to lock the support part (102) with the bed (104) by passing through the one or more support part slots and the one or more bed slots, wherein the one or more fasteners (108) are configured to lock the support part (102) against the bed (104) holding the skin graft on the placement area (106).

11. The method as claimed in claim 8, wherein the lifting element is provided on a first ring that is locked securely with the bed (104), wherein the lifting clement comprises four springs mounted on the first ring at 90 degrees apart (i) to keep the skin graft intact on the placement area (106), and (ii) to push the support part (102) gradually upward during unlocking of the support part (102) to perform the second cut.
